SEQUENCE LISTING (6 sequences)

SEQ ID NO: 1 - DNA, 2699 bp, Homo sapiens
  TATA_signal (1311)..(1325)
tcaggctggcctcaaactcctgacctcgtgatcggtgcgcctcggcctcccaaaatgctggggttacaggcgtgagccactgcacccggccccaaaatctaacgtttgttatcactgttgttttagaaaagaaaggacattttaatgcaacccatgcccatctgtaggggctgaaggtccctctgaggtgattgataaagctgaaaaaagagactatgggaagagaagaggaatattatcaattagctattttagaagcagggcaaactatgagctaggtattcctataaggaaatgaaataatttactaaaaaacatacagcaagaaaataatagagctggagttttctgatttttctgagtccagaatttgtttcactgttctcatttcagctctctctaacaggatgaatgaccttgatttcagaatccccaagcaggtggtgagatccaaaactgagacaaaagaaacggggctgttccaaaaaaaaagctaggtggcaggtgtctaacatgccagggagctaaaacagagtgtgtgagtttcagcagcaggttgaatttagaatggggaaggagaccagaggagacgccagacaggatgactttgtcccattggcctggaggcagccccatgtttctccacccctcatatcactcccagtttgtaatagtatctttgaatgacgatctgattaaggtccgtctcctccattagtccacaagtttcgggggtacatctactttgctcatttccatatccccagagtctagcacaaggcctggtacatagtaggtgctcaataaatatgttagatgaaaggaagataacacctctatgtactagcagtgagactccaggcatgcaatttctctctgtccttcagtcccttcatctcaaggtttaatttaaatatggtaacgcctgtatgcaactcccagcatccagtaggcactcactaaacacagttctccaccctccttttttcctctgcccctccctcggttttcccactacttcctgcatggtgacacacccatagtttggagccataaaacccaacccaggttggactctcacctctccagccccttctgctccggccctgtcctcaaattggggggctgatgtccccatacacctggctctgggttcccctaaccccagagtgcaggactaggacccgagtggacctcaggtctggccaggtcgccattgccatggagacagcaacagtccccagccgcgggttccctaagtgactggttactctttaacgtatccacccaccttgggtgattagaagaatcaataagataaccgggcggtggcagctggccgcactcaccgccttcctggtggacgggctcctggtggctgtgctgctgctgtgagcgggcccctgctcctccatgcccccagctctccggctgggtgggcttggccatggtcagcgtgaacgcgcccctcggggctccagtggagagttcttacggtaagtggggctgggggaagactggacagggcgggactgcggtcagctttgggaggccatgggacacctccccgtgtgtttcttacgggcccaaagctcctcctggagctcctctggaagggcaggaagccccacggaggcaatgtgaccgcttccctaagctctcaagccggggcatggcctgcttggtgcgagaagtgctgggaggggccgagaagccctggggcagaggcttggctcattttgtctgtccagaggccttcctgggaatgcatctcaggggtctttccaaaaccccacagagggtggggcttggaagcaccgtcctgtttcgatgcggggcaaattgaggtccaccaggagaggcttgctgggcctaggtcacgttgctggtgcattatcaagctgggcgtgggaccgggcagctcggtcgctccgcacctccgttggctctggataatggggaggaggtagaaagcgccgcgcagggtggagcgttggaaaatgagaatatcttgtgcggacgtgtgaaactcagatgcagggctagaaagcatgtgttactgagagtgaaaggctcaggctcactccttagggtgacccccacattggaagcaacccaatgtccctcagcagagggaagacgggtaaataaagcgtgagacgtctttacagtggaatgctatcccattgttcaaggaaacaacgcacatctgtgtgtgacagggaagaacctctcagttaaattaagtgaaaacggtacactgaagaactgtgtagttgctgatagcgaaacggatatagatgtatatatgtacacacgcgtatacgtgtacattccacgcatagaaaattcgtggaatgacacaataaaattggtgacaggtttgtttctgaggagggaaattcaagagaatgttttatcttttggggacacattttggattttttaaaagttgggtgccttttataaacctgttcaaatcacacacccgcacagaacttggtctctggaatccagcagacctcggggtctagttgtgactctgtcacacggctcaacattcacgtgcctcttgctttctttctgaagtggagacaggactaggatgtgtcttaagatcatggtggatgccgtggggaacaaggatgtaaagcccttgataccacgtgcattgcaaagacacaatcaacatttatgtc

SEQ ID NO: 2 - protein, 16 aa, Homo sapiens
MVSVNAPLGAPVESSY

SEQ ID NO: 3 - DNA, 2588 bp, Homo sapiens
  TATA_signal (1279)..(1294)
cggtgcgcctcggcctcccaaaatgctggggttacaggcgtgagccactgcacccggccccaaaatctaacgtttgttatcactgttgttttagaaaagaaaggacattttaatgcaacccatgcccatctgtaggggctgaaggtccctctgaggtgattgataaagctgaaaaaagagactatgggaagagaagaggaatattatcaattagctattttagaagcagggcaaactatgagctaggtattcctataaggaaatgaaataatttactaaaaaacatacagcaagaaaataatagagctggagttttctgatttttctgagtccagaatttgtttcactgttctcatttcagctctctctaacaggatgaatgaccttgatttcagaatccccaagcaggtggtgagatccaaaactgagacaaaagaaacggggctgttccaaaaaaaaagctaggtggcaggtgtctaacatgccagggagctaaaacagagtgtgtgagtttcagcagcaggttgaatttagaatggggaaggagaccagaggagacgccagacaggatgactttgtcccattggcctggaggcagccccatgtttctccacccctcatatcactcaccagtttgtaatagtatctttgaatgacgatctgattaaggtccgtctcctccattagtccacaagtttcgggggtacatctactttgctcatttccatatccccagagtctagcacaaggcctggtacatagtaggtgctcaataaatatgttagatgaaaggaagataacacctctatgtactagcagtgagactccaggcatgcaatttctctctgtccttcagtcccttcatctcaaggtttaatttaaatatggtaacgcctgtatgcaactcccagcatccagtaggcactcactaaacacagttctccaccctccttttttcctctgcccctccctcggttttcccactacttcctgcatggtgacacacccatagtttggagccataaaacccaacccaggttggactctcacctctccagccccttctgctccggccctgtcctcaaattggggggctgatgtccccatacacctggctctgggttcccctaaccccagagtgcaggactaggacccgagtggacctcaggtctggccaggtcgccattgccatggagacagcaacagtccccagccgcgggttccctaagtgactggttactctttaacgtatccacccaccttgggtgattagaagaatcaataagataaccgggcggtggcagctggccgcactcaccgccttcctggtggacgggctcctggtggctgtgctgctgctgtgagcgggcccctgctcctccatgcccccagctctccggctgggtgggcttggccatggtcagcgtgaacgcgcccctcggggctccagtggagagttcttacggtaagtggggctgggggaagactggacagggcgggactgcggtcagctttgggaggccatgggacacctccccgtgtgtttcttacgggcccaaagctcctcctggagctcctctggaagggcaggaagccccacggaggcaatgtgaccgcttccctaagctctcaagccggggcatggcctgcttggtgcgagaagtgctgggaggggccgagaagccctggggcagaggcttggctcattttgtctgtccagaggccttcctgggaatgcatctcaggggtctttccaaaaccccacagagggtggggcttggaagcaccgtcctgtttcgatgcggggcaaattgaggtccaccaggagaggcttgctgggcctaggtcacgttgctggtgcattatcaagctgggcgtgggaccgggcagctcggtcgctccgcacctccgttggctctggataatggggaggaggtagaaagcgccgcgcagggtggagcgttggaaaatgagaatatcttgtgcggacgtgtgaaactcagatgcagggctagaaagcatgtgttactgagagtgaaaggctcaggctcactccttagggtgacccccacattggaagcaacccaatgtccctcagcagagggaagacgggtaaataaagcgtgagacgtctttacagtggaatgctatcccattgttcaaggaaacaacgcacatctgtgtgtgacagggaagaacctctcagttaaattaagtgaaaacggtacactgaagaactgtgtagttgctgatagcgaaacggatatagatgtatatatgtacacacgcgtatacgtgtacattccacgcatagaaaattcgtggaatgacacaataaaattggtgacaggtttgtttctgaggagggaaattcaagagaatgttttatcttttggggacacattttggattttttaaaagttgggtgccttttataaacctgttcaaatcacacacccgcacagaacttggtctctggaatccagcagacctcggggtctagttgtgactctgtcacacggctcaacattcacgtgcctcttgctttctttctgaagtggagacaggactaggatgtgtcttaagat

SEQ ID NO: 4 - protein, 16 aa, Homo sapiens
MVSVNAPLGAPVESSY

SEQ ID NO: 5 - DNA, 311 bp, Homo sapiens
  TATA_signal (124)..(138)
agtggacctcaggtctggccaggtcgccattgccatggagacagcaacagtccccagccgcgggttccctaagtgactggttactctttaacgtatccacccaccttgggtgattagaagaatcaataagataaccgggcggtggcagctggccgcactcaccgccttcctggtggacgggctcctggtggctgtgctgctgctgtgagcgggcccctgctcctccatgcccccagctctccggctgggtgggcttggccatggtcagcgtgaacgcgcccctcggggctccagtggagagttcttacggt

SEQ ID NO: 6 - protein, 16 aa, Homo sapiens
MVSVNAPLGAPVESSY

(19) United States
(12) Patent Application Publication
(10) Pub. No.: US 2004/0038219 A1
(43) Pub. Date: Feb. 26, 2004

(54) Method for treating diabetes

(76) Inventors: Silvia Boj Fernandez, Barcelona (ES); Torben Hansen, Hellerup (DK); Sara Kristine Frederiksen, Frederiskberg (DK); Maria Lajer Jensen, Frederiskberg (DK); Oluf Borbye Pedersen, Lyngby (DK); Marcelina Parrizas, Barcelona (ES); Miguel Angel Maestro, Barcelona (ES); Jan Lebl, Praha (CZ); Stepanka Pruhova, Karlovy Vary (CZ); Jorge Pedro Ferrer, Sant Cugat del Valles (ES)

Correspondence Address: Reza Green, Esq., Novo Nordisk of North America, Inc., Suite 6400, 405 Lexington Avenue, New York, NY, 10174-6401, US

(21) Appl. No.: 10/228,368

(22) Filed: Aug. 13, 2002

Related U.S. Application Data
(60) Provisional application No. 60/325,271, filed on Sep. 27, 2001.

(30) Foreign Application Priority Data
Aug. 17, 2001 (EP) EP 01610085.1

(51) Int. Cl. C12Q 1/68; A61K 31/00

(52) U.S. Cl. 435/6; 514/1

(57) ABSTRACT

The invention regards a screening method for screening for modulators of the expression of hnf4α from the hnf4α P2 promoter region, which method comprises the steps of obtaining a transcription reporter system comprising a nucleotide construct comprising a reporter gene under control of the hnf4α P2 promoter region corresponding to nucleotide 1 to 1447 of the sequence shown in the Sequence listing as SEQ ID No. 1 or a fragment thereof, contacting the transcription reporter system with a putative modulator and assaying for a change in the level of expression of the reporter gene.

### CROSS REFERENCE TO RELATED APPLICATIONS

This application claims priority under 35 U.S.C. §119 of European application EP 01610085.1 filed Aug. 17, 2001, and of U.S. provisional application No. 60/325,271 filed Sep. 27, 2001, the contents of which are hereby incorporated by reference.

### BACKGROUND OF THE INVENTION

Terminally differentiated pancreatic β-cells possess a highly specialized apparatus designed to sense extracellular concentrations of glucose, other metabolites, and hormones. This information is processed to couple the synthesis and secretion of insulin to the organism's demands. Despite growing knowledge derived from mouse genetic studies indicating which transcriptional regulators are required to complete discrete steps in pancreatic development, much less is known regarding the transcription factors needed to ensure the specialized functions of differentiated β-cells once these are formed. Recent human genetic studies have pointed to a set of genes whose role may be relevant to this function. Thus, humans with heterozygous mutations in the genes encoding hepatocyte nuclear factors (HNF) 1α, 1β, and 4α, as well as pancreatic islet duodenum homeobox PDX1/IDX1/IPF1 develop a diabetic phenotype resulting from β-cell dysfunction (Maturity Onset Diabetes of the Young, MODY) (Yamagata, K. et al. (1996) Nature 384, 455-458 and Yamagata, K. et al. (1996) Nature 384, 458-460).

Perhaps the best-characterized MODY defect is that resulting from mutations in the gene encoding hnf1α, an a typical homeodomain protein. A hallmark of the pathophysiology of human hnf1α-deficiency (MODY3) and hnf1α-null mutant mice is defective β-cell glucose sensing. This results at least in part from reduced aerobic glycolysis and possibly mitochondrial metabolism although the precise molecular defects are unknown. It is likely that several islet-cell enriched genes are involved, including the glut2 glucose transporter, liver type pyruvate kinase (pklr), and aldolase B. Mice lacking hnf1α are nevertheless capable of forming islet structures without a conspicuous decrease of β-cell mass or insulin content. Thus, the primary role of hnf1α in the endocrine pancreas may be to ensure fine-tuning of insulin output in response to metabolic demands.

Decreased glucose-induced insulin release has also been shown to underlie hnf4α-deficient diabetes (MODY1) (Byrne, M. M. et al (1995) Diabetes 44, 699-704). The related phenotype of MODY subtypes could plausibly stem from the existence of a pancreatic network formed by different transcription factors involved in MODY. Several lines of evidence have indeed revealed a regulatory hierarchy whereby hnf4α controls the transcription of hnf1α in embryonic liver cells and cultured hepatocyte cell lines (Kuo, C. J. et al. (1992) Nature 355, 457-461 and Li, J. et al (2000) Genes Dev. 14, 464-474). Because the diabetogenic defect in MODY lies in β-cells, rather than in hepatocytes, it was speculated that similar interactions between hnf4α and hnf1α occur in β-cells, although this has not yet been experimentally addressed.

It is likely that hnf1α- and hnf4α-dependent transcriptional regulation in β-cells has distinct properties relative to other tissues, as human heterozygous mutations result in selective defective β-cell function, with only minor or no conspicuous abnormalities in other tissues where these genes are expressed (Byrne, M. M. et al. (1996) Diabetes 45, 1503-1510). Furthermore, earlier work has shown that two target promoters, glut2 and liver type pyruvate kinase (pklr), are occupied in vivo by hnf1α in diverse mouse tissues, but only require hnf1α for gene activity in islet cells (14).

Studies performed primarily in hepatocyte lineages have revealed a transcriptional regulatory hierarchy whereby hnf4α controls hnf1α transcription (Kuo, C. J. et al (1992) and Li, J. (2000), ibid). Because diabetes in humans with hnf1α and hnf4α deficiency (MODY3 and MODY1, respectively) results from a similar insulin-secretory abnormality (Byrne, M. M. et al (1995) and Byrne, M. M. et al (1996), ibid) it has been inferred that the same regulatory hierarchy is operative in β-cells (Froguel, P. et al (1999) Trends Endocrinol. Metab 10, 142-146).

### SUMMARY OF THE INVENTION

In this study we have identified a novel transcription factor circuit that is controlled by hnf1α specifically in differentiated pancreatic endocrine and exocrine cells. These studies place hnf4α downstream of hnf1α, in sharp contrast to what is known to occur in liver (Kuo, C. J. et al (1992), ibid), and reveal that hnf1α-dependence is mediated through a tissue-specific alternate hnf4α promoter. Hnf1α is also shown to control pancreatic expression of hnf4γ and hnf3γ. Chromatin immunoprecipitations (ChIP) were used to map transcription factor-promoter interactions in vivo in mouse tissues, providing a partial structure of the regulatory circuit. Furthermore, the circuit is shown to be switched on as differentiated pancreatic cells arise in embryonic development. The results uncover a regulatory strategy employed by hnf1α in pancreatic cells, and reveal that pancreatic exocrine and endocrine cells do not only share common cellular precursors, but also a tissue-specific genetic program that is likely to be involved in the control of differentiated cellular functions.

A screening method for screening for modulators of the expression of hnf4α from the hnf4α P2 promoter region, which method comprises the steps of a) obtaining a transcription reporter system comprising a nucleotide construct comprising a reporter gene under control of the hnf4α P2 promoter region corresponding to nucleotide 1 to 1447 of the sequence shown in the Sequence listing as SEQ ID No. 1 or a fragment thereof, b) contacting the transcription reporter system with a putative modulator and c) assaying for a change in the level of expression of the reporter gene.

The present invention also provides a modulator of the expression of hnf4α identified by a method according to the present invention.

The present invention also provides a method for treating diabetes in an animal, which method comprises the step of modulating the expression of hnf4α from the hnf4α promoter 2.

The present invention also provides a “method for treating diabetes in an animal, which method comprises the step of “administering a β cell specific hnf4α agonist” to an animals suffering from diabetes”.

SEQ ID No. 1 originates from accession no. AL117382.28 (nucleotides 86701 to 89400). Exon 1D is situated from nucleotide 1448 to 1496. The intron/exon boundary consensus site (gt) is situated at nucleotides 1497 and 1498.

SEQ ID No. 2 also originates from accession no. AL117382.28 (nucleotide 86733 to 89320).

SEQ ID No. 3 also originates from accession no. AL117382.28 (nucleotide 88199 to 87889) including the hnf1α binding site covering position 79 to 93, the TATA signal and exon ID. The mutation in the Czech family is a mutation of G in position 80 to an A.

### BRIEF DESCRIPTION OF THE FIGURES

FIG. 1

Pancreatic-Specific hnf1α-Dependence of hnf4α

RT-PCR analysis of hnf4α mRNA in purified tissues of hnf1α^{-/-} and ^{+/+} mice PCR was carried out with oligonucleotides amplifying a region encompassing exon 2 and 3 of hnf4α mRNA. β-actin, tbp or hprt were coamplified to control for cDNA amount and PCR efficiency. Arrowheads indicate expected position of hnf4α PCR products.

FIG. 2

Pancreatic hnf4α mRNA is Transcribed from a Tissue-Specific Alternate Promoter

(A) Alignment of human and mouse genomic sequences encoding the 5′ end of pancreatic hnf4α mRNA (exon 1D), and its 5′ flanking region. ↓: 5′ end of the RACE product, ▾∇: prediction of transcription initiation using TSSW and NPPW programs (37), [symbol] : 5′ end of hnf4α7 cDNA sequence (Nakhei, H. et al (1998) Nucleic. Acids. Res. 26, 497-504). •: exon-intron boundary. A horizontal line indicates the predicted initiator codon. The hnf1-binding site is boxed.

(B) RT-PCR analysis of hnf4α transcripts containing either exon 1A or exon 1D sequences in tissues from hnf1α^{-/-} and ^{+/+} mice. Arrowheads indicate expected position of hnf4α PCR products.

(C) RT-PCR analysis of hnf4α mRNA in pancreatic endocrine and exocrine fractions from hnf1α^{-/-} and ^{+/+} mice. Glut2 and amylase mRNA were assayed to indicate tissue purity.

(D) Schematic indicating the genomic position of exon 1D and P2 promoter relative to the known hnf4α promoter (P1).

(E) ChIP analysis using antibodies recognizing acetylated histones H3 and H4 (AcH3, AcH4), indicating tissue-specific, hnf1α-dependent hyperacetylation of P2 in pancreatic islets. The gapdh promoter is used as a control, representing a hyperacetylated chromatin region which is unaffected by hnf1α. PI: preimmune serum. Input 1:50, 1:150 1:100, 1:300: diluted DNA purified prior to ChIP to indicate expected amplification patterns in the absence of enrichment of specific DNA fragments.

FIG. 3

Hnf1α Occupies hnf4α P2 Promoter DNA in Vitro and in Vivo

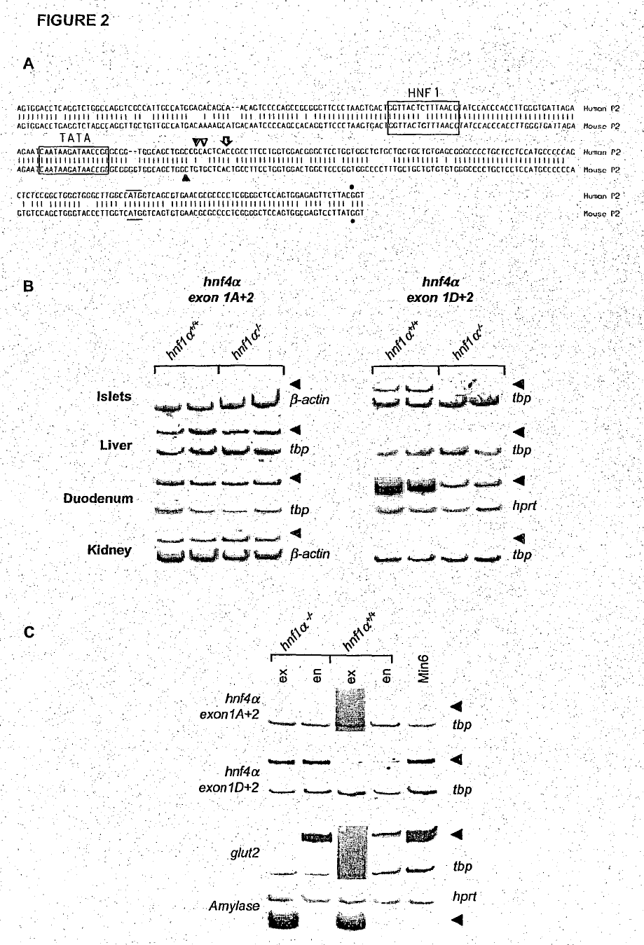

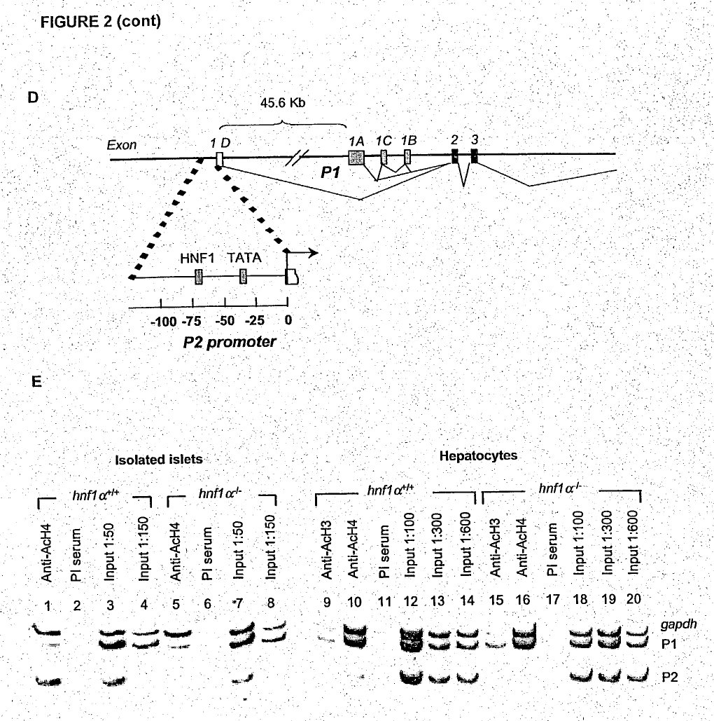

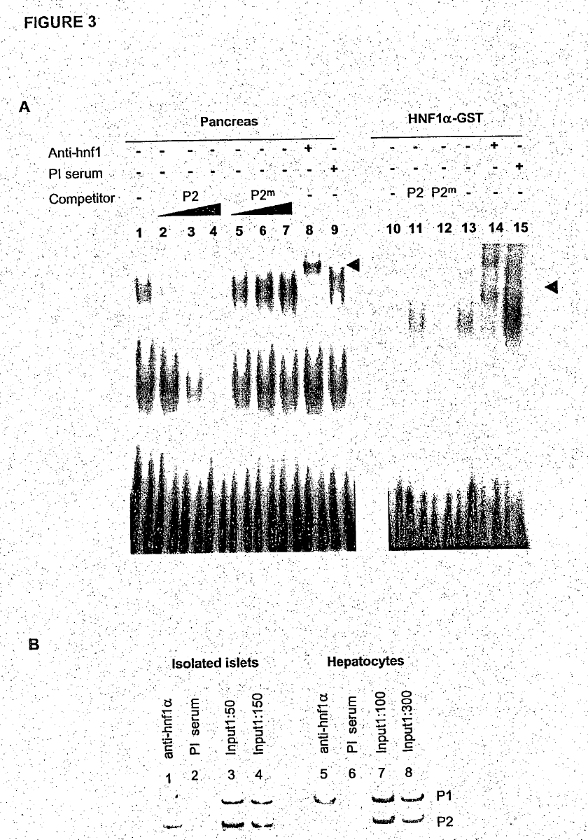

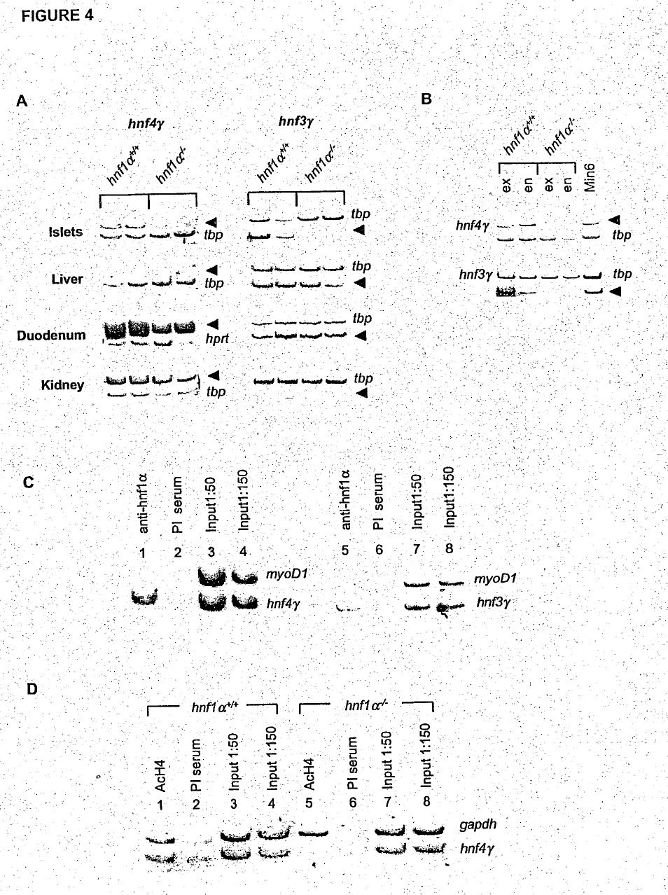

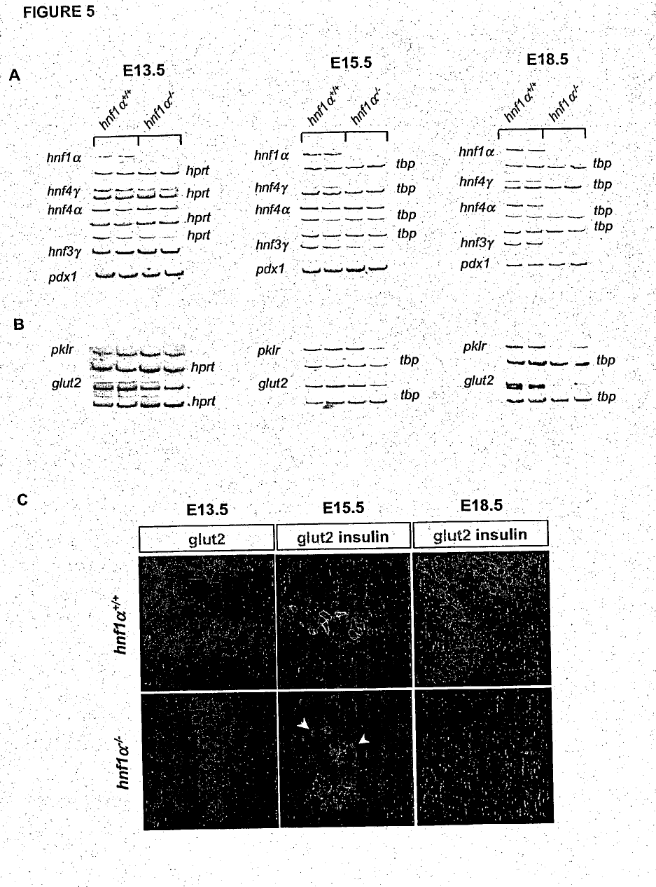

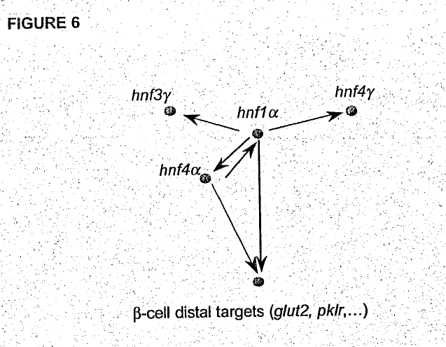

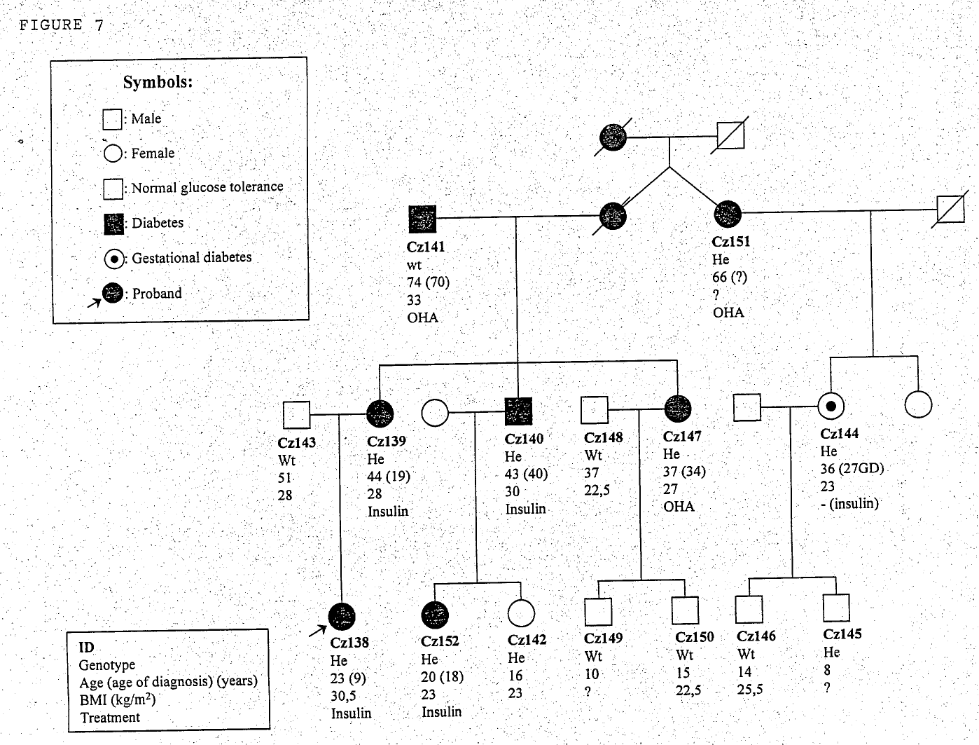

(A) Left panel. EMSA showing interaction of a pancreatic nuclear complex to an oligonucleotide containing the site boxed in FIG. 2A. Binding is competed by excess (x2 to x200) unlabeled probe but not by a oligonucleotide with two single base substitutions. Anti-hnf1α antibody but not preimmune serum supershifts the complex. Analogous results were observed using recombinant GST-hnf1α (right panel) and purified islet and mouse hepatocyte nuclear extracts and (not shown).

(B) ChIP assays indicate that hnf1α occupies the endogenous hnf4α P2 promoter in islets (lane 1, compare with preimmune serum in lane 2 and input DNA), whereas in liver it contacts the P1 promoter (lane 5). Input 1:50, 1:150, 1:100, 1:300: diluted DNA purified prior to immunoprecipitation.

FIG. 4

Hnf1α-Dependence of hnf4γ and hnf3γ in Pancreatic Cells

(A) RT-PCR analysis of hnf4γ and hnf3γ mRNA in tissues from hnf1α^{-/-} and ^{+/+} mice. Arrowheads indicate expected position of hnf PCR products.

(B). RT-PCR analysis of hnf4γ and hnf3γ mRNA in pancreatic endocrine and exocrine tissues from hnf1α^{-/-} and ^{+/+} mice (see FIG. 2C for more details and tissue-specific markers).

(C) ChIP analysis indicating that hnf1α directly occupies the hnf4γ promoter and hnf3γ enhancer in vivo (right panel). See FIGS. 2D and 3B for more details.

(D) ChIP analysis using antibodies recognizing acetylated histones H3 and H4 (AcH3, AcH4) indicate that hnf4γ promoter chromatin is enriched in hyperacetylated histone tails, and this is dependent on hnf1α function. See footnotes in FIGS. 2 and 3 for further details.

FIG. 5

Ontogeny of the Pancreatic hnf1α-Dependent Genetic Program

(A) and (B) Reverse transcription PCR analysis of dissected pancreatic tissue at embryonic ages E13.5, E15.5, and E18.5 reveals that hnf1α-dependence of hnf4α, hnf4γ, hnf3γ, glut2 and pkl3 is established in parallel with the surge of differentiated pancreatic cells. Exon 1D-specific primers were used for hnf4α amplification.

(C) Immunofluorescence analysis of glut2 (shown in green) in timed embryonic pancreas. Note that loss of glut2 in hnf1α^{-/-} embryos is first elicited at E15.5 in some insulin positive cells (red) (white arrowheads). At E18.5 all insulin-positive cells are negative for glut2 in hnf1α^{-/-} embryos.

FIG. 6

Model Representing a Pancreatic β-Cell Specific Complex Transcriptional Regulatory Circuit Controlled by hnf1α

Regulatory interactions are derived from this study and Miquerol, L. et al (1994) J. Biol. Chem. 269, 8944-8951 indicating direct interactions between hnf4 and the pklr 5′flanking region.

FIG. 7

Pedigree of the Czech Family cz138

Genotype “wt” means that the person does not carry the mutation of G in position 80 in SEQ ID No. 3 to an A, while genotype“he” means that the person carries said mutation. All adult members of the family, which carries the mutation have diabetes or gestational diabetes (one person) with debut at a young age and often with a need for insulin treatment. Two young carriers of the mutation have not yet been examined for diabetes. One person of the oldest generation has ordinary type 2 diabetes with a debut at the age of 70.

### DEFINITIONS AND ABBREVIATIONS

HNF: hepatocyte nuclear factor. MODY: maturity onset diabetes of the young. RT-PCR: reverse transcription-polymerase chain reaction. ChIP: chromatin immunoprecipitation. OHA: oral hyperglycaemia agents.

### DETAILED DESCRIPTION OF THE INVENTION

The studies performed in connection with the present invention and described herein show that in-pancreatic cells hnf1α occupies an alternate hnf4α promoter (hnf4α P2 promoter or simply P2) located more than 45 Kb away from the one previously characterized in liver cells, and acts as an obligate factor to induce nucleosomal hyperacetylation and gene activity. Moreover, the results indicate that the pancreas represents the sole organ where P2 is the predominant hnf4α promoter throughout embryonic and postnatal development. These findings have several interesting implications. Because this epistatic interaction occurs in the cells which are involved in the pathogenesis of MODY, it is likely to be fundamental to the common insulin secretory phenotype occurring in hnf1α and hnf4α-deficiency. The fact that hnf4α is downstream of hnf1α in these cells offers a potential mechanism to bypass the hnf1α-block to correct the insulin secretory defects of hnf1α-deficient diabetes. Importantly, attempts to manipulate the expression of hnf4α in pancreatic cells should be directed at a site located more than 45 Kb away from the previously known hnf4α promoter. Furthermore, the finding that hnf4α is transcribed from a distinct promoter in pancreatic cells provides a new candidate sequence to search for variants causing MODY1 diabetes or underlying the chromosome 20q susceptibility region for polygenic Type 2 diabetes identified in several genome scans (reviewed in Permutt, M. A. et al, (2000) Trends Endocrinol. Metab 11, 383-393).

At least two more transcriptional activators, hnf4γ and hnf3γ, are downstream of hnf1α in pancreatic cells. The precise role of these activators in pancreatic β-cells is currently unknown. For hnf3γ genetic inactivation in mice has revealed no pancreatic phenotype (Kaestner, K. H. et al (1998) Mol. Cell Biol. 18, 4245-4251). However, the consequences of hnf3γ deficiency in hnf1α^{-/-} islets cannot be predicted from the hnf3γ^{-/-} phenotype if there is functional redundancy between hnf1α-dependent transcriptional regulators. Perhaps most important at this time is the finding that hnf1α orchestrates a broad transcription factor circuit which is specific for pancreatic cells (FIG. 6). Very recent data supports the notion that regulation of subsidiary transcription factors may be a modus operandi of hnf1α function. Thus, hnf1α-control of cholesterol metabolism enzymes in the liver was shown to be mediated through regulation of a transcriptional hierarchy whereby hnf1α activates the nuclear receptor FXR-1 gene, which in turn transactivates Shp-1, a repressor of cholesterol 7-hydroxylase transcription.

The results presented here suggest a complex structure of the pancreatic hnf1α-dependent regulatory circuit. ChIP analysis indicates that hnf1α directly occupies the promoter regions of subsidiary transcription factors. Previous work has shown that hnf1α also directly occupies the promoter regions of non-transcription factor genes (e.g. glut2 and pklr) (Parrizas, M. et al (2001)). Based on these findings we postulate a circuit model whereby rather than a simple lineal hierarchy in which hnf1α acts either as the sole essential activator binding to a given distal target promoter, or indirectly through regulation of a single intermediary activator, transcription of targets such as glut2 and pklr could be dependent on hnf1α inasmuch as they require the function of both hnf1α and its downstream transcription factors. In support for this proposal, ChIP results using two different hnf4α antisera indicate coocupation of glut2 and pklr promoter region chromatin by hnf4α along with hnf1α (unpublished observations Marcelina Párrizas and Jorge Ferrer). Thus, silencing of glut2 and pklr genes selectively in islet cells from hnf1α^{-/-} mice could result from the combined tissue-restricted failure of a set of transcription factors required for its expression, including hnf1α, hnf4α and plausibly others.

The diabetic phenotype in humans with MODY3 typically appears during the second decade of life (Lehto, M. et a/. (1997) J. Clin. Invest 99, 582-591). Similarly, hnf1α^{-/-} mice do not exhibit defective pancreatic organogenesis, and develop manifest hyperglycemia only several weeks after birth. This does not prove however that the requirement for hnf1α in the pancreas is restricted to postnatal cells. The studies shown here reveal that hnf1α-dependence of a set of pancreatic genes is activated in early differentiated cells of the embryonic pancreas. The switch between hnf1α-independent to hnf1α-dependent states was elicited most accurately for glut2. Thus, the requirement for hnf1α to express glut2 is observed shortly after insulin-producing cells arise. These findings suggest that hnf1α is either essential to initiate glut2 expression in differentiated β-cells, in which case glut2 staining in some early insulin-positive cells reflects a long residence time of proteins activating glut2 in precursor cells or of glut2 itself, or alternatively, hnf1α may be dispensable to initiate glut2 transcription in β-cells, but required to maintain its expression in differentiated cells.

Without wishing to be bound by any theory, the critical function of hnf1α in the pancreas thus appears to be to deploy a genetic program in cells that have already committed to a differentiated pancreatic fate. The program shares common transcription factors in both endocrine and exocrine pancreatic cell types, but possesses cell-specific distal target readouts most likely determined by additional cell-specific regulatory factors. Although the possible physiological role of the circuit in exocrine cells has not yet been examined, it appears likely that the ultimate mission in insulin-producing cells is to support the expression of genes like glut2 and pklr involved in highly specialized functions such as glucose sensing. These results allow us to place the role of the hnf1α-dependent transcription circuit in pancreatic embryonic development at a discrete stage subsequent to those known to be dependent on other transcription factors including those involved in early pancreatic bud development (e.g. pdx1 or hlxb-9), proendocrine commitment within pluripotential pancreatic cells (e.g. ngn3), refinement to a β-cell fate (e.g. pax4 or nkx6.1) or early differentiation of β-cells (e.g. nkx2.2).

A mutation in the human hnf4α P2 promoter region has been identified in a Czech family (cz138), where seven family members suffering from MODY are carriers of the mutation, while two young family members are carrying the mutation but are still glucose tolerant (see FIG. 7). The mutation is a change in the hnf1α binding site in the hnf4α P2 promoter region, namely a mutation of G in position 80 in SEQ ID No. 3 to an A. This finding confirms that hnf4α P2 promoter region also seems to be important in humans and that a defect in this promoter, leading to decreased expression of hnf4α, is tightly associated with the development of MODY.

The present invention provides a screening method for screening for modulators of the expression of hnf4α from the hnf4α P2 promoter region, which method comprises the steps of

a) obtaining a transcription reporter system comprising a nucleotide construct comprising a reporter gene under control of the hnf4α P2 promoter region corresponding to nucleotide 1 to 1447 of the sequence shown in the Sequence listing as SEQ ID No. 1 or a fragment thereof,

b) contacting the transcription reporter system with a putative modulator and

c) assaying for a change in the level of expression of the reporter gene.

The person skilled in the art will know how to set up such screening methods. The transcription reporter system could for instance comprise a cell, such as an bacteria cell, for instance an Eschericia coli cell, a eucaryotic cell, for instance a Saccharomyces cerevisiae cell, a Chinese hamster ovary cell, a pancreatic β cell or any cell capable of harbouring a nucleotide construct comprising a reporter gene. The transcription reporter system may also be a solution comprising the necessary parts for conducting transcription of the reporter gene, including any transacting factors of interest. The person skilled in the art will know how to set up such a transcription reporter system.

The transcription reporter system may measure the activity from the promoter by one or more of several ways of measuring such activity as it is known in the art such as by measuring the level of transcript or by measuring the amount of polypeptide encoded by the reporter gene. The manner in which the activity of the promoter is measured depends on the nature of the reporter gene.

In one embodiment, the transcription reporter system comprises hnf1α, for instance by comprising a nucleotide construct encoding hnf1α under control of a promoter which can be expressed in the given transcription reporter system, which construct might be of endogenous or exogenous origin, for instance a result of genetic engineering, or by having hnf1α added to the system as a polypeptide.

The reporter gene is a gene the expression of which can be detected by any suitable means. The person skilled in the art is aware of many such genes, for instance lacZ from Eschericia coli, and will be able to choose such a gene according to preferences. The nature of the transcription reporter system will naturally depend on the nature of the reporter gene. All this is well known in the art.

The hnf4α P2 promoter region is be the 5′ flanking region of pancreatic hnf4α mRNA exon 1D, or any fragment or homologue thereof. Such a fragment may for instance be inserted into another promoter to create a hybrid promoter. A homologue is the 5′ flanking region of pancreatic hnf4α mRNA exon 1D, or a fragment thereof, in which one or more nucleotides are substituted with a different nucleotide. Such homologues may for instance arise due to genetic diversion within the species or between the species or may be artificially introduced for a reason, such as for eliminating certain binding sites, creating new binding sites or otherwise altering the function of the promoter. This way the screening may be directed specifically at finding modulators of the expression of hnf4α from the hnf4α P2 promoter region, which modulators modulate in a specific manner related to the kind of nucleotide substitution introduced.

Assaying of the level of expression of the reporter gene will be dependent on the reporter gene The level of expression of the reporter gene may be assayed for instance by measuring the amount of transcript of the reporter gene, such as by Northern Blots or other ways known in the art, and in such a case the reporter gene may for instance be hnf4α itself, or by measuring an enzymatic effect of the reporter gene or by any other way known by the person skilled in the art to be relevant for a given reporter gene.

As the person skilled in the art will recognize, a change in the level of expression of the reporter gene is a change that is statistically significant for the given system.

In one embodiment the hnf4α P2 promoter region corresponds to nucleotide 1 to 1447 of the sequence shown in the Sequence listing as SEQ ID No. 1, or a fragment thereof, a hnf1α binding site. The binding site of hnf1α may comprise nucleotides 79 to 93 of SEQ ID No. 3, but may also be a mutated version of this binding site, such as being a fragment of this sequence or by comprising substitutions of one or more nucleotides as long as hnf1α is capable of binding to it. The person skilled in the art will know how to determine such a binding, for instance by gel retardation assays.

A fragment of hnf4α P2 promoter region corresponding to nucleotide 1 to 1447 of the sequence shown in the Sequence listing as SEQ ID No. 1 comprising a hnf1α binding site may be inserted into another promoter to create a hybrid promoter, where the expression form said promoter thereby will become modulated by hnfα1. Such a promoter may for instance be the promoter into which the binding site for hnf1α is inserted.

The present invention also provides a modulator of the expression of hnf4α identified by a screening method according to the present invention. Such a modulator may be capable of affecting the hnf4α P2 promoter directly by actually binding to the promoter region itself, or it may effects its modulating effects indirectly, for instance by binding to another transcription factor involved in the transcription from the hnf4α P2 promoter or in any other way. The manner in which said modulator asserts its effects shall not be construed as limiting the present invention. The modulator's effect on the hnf4α P2 promoter should be a specific effect meaning that it does not act as a general modulator of the common transcriptional machinery but that it is specifically directed at modulating the transcription from the hnf4α P2 promoter region

The present invention also provides a method for treating diabetes in an animal, which method comprises the step of modulating the expression of hnf4α in pancreatic cells.

In one embodiment of method for treating diabetes according to the present invention, the method comprises the step of modulating the expression of hnf4α from the hnf4α P2 promoter corresponding to nucleotide 1 to 1447 of the sequence shown in the Sequence listing as SEQ ID No. 1.

In one embodiment of method for treating diabetes according to the present invention, the modulation results in an increase of the expression of hnf4α.

In one embodiment of method for treating diabetes according to the present invention the modulation is effected via the hnf1α binding site corresponding to nucleotides 79 to 93 of SEQ ID No. 3 or a fragment thereof to which hnf1α is capable of binding.

In one embodiment, the animal to be treated is a mammal and in a further embodiment, the animal is a human being.

In one embodiment of method for treating diabetes according to the present invention the animal suffers from MODY (maturity onset diabetes of the young).

In one embodiment of method for treating diabetes according to the present invention the animal suffers from MODY1.

In one embodiment of method for treating diabetes according to the present invention the animal suffers from type 2 diabetes.

In one embodiment of method for treating diabetes according to the present invention the modulation is achieved by administering a modulator according to the present invention to the animal in an effective dose.

The present invention also provides the use of a modulator according to the present invention for treating diabetes in an animal. In one embodiment the animal is a mammal and in a further embodiment the animal is a human being.

The present invention also provides a diagnostic method for screening for diabetes comprising: a) obtaining a sample nucleic acid from an animal; and b) analyzing the nucleic acid to detect a mutation in the hnf4α P2 region as shown in SEQ ID No. 1, wherein the mutation results in a lesser degree of expression of hnf4α, wherein a mutation in the hnf4α P2 promoter region is indicative for a propensity for diabetes.

In one embodiment of said diagnostic method the hnf4α P2 promoter region corresponds to nucleotide 1 to 1447 of the sequence shown in the Sequence listing as SEQ ID No. 1.

In one embodiment of said diagnostic method the hnf4α P2 promoter region comprises a hnf1α binding site.

In one embodiment of said diagnastic method the hnf4α P2 promoter region comprises the region corresponding to nucleotides 79 to 93 of the sequence shown in the sequence listing as SEQ ID No. 1.

In one embodiment of said diagnostic method the mutation is a mutation of G in the position corresponding to position 80 in SEQ ID No. 3 to an A.

In one embodiment of said diagnostic method the animal is a mammal and in a further embodiment the animal is a human being.

Those skilled in the art will readily recognize that it is within the scope of the present invention to analyse said samples for more than one mutation in the hnf4α P2 region or additionally analyse said samples for other mutations in the hnf4α P2 region of interest, or indeed for mutations in other genes associated with diabetes or otherwise of interest.

In one embodiment of said diagnostic method, the sample nucleic acid is obtained from a subject, DNA (in particular genomic DNA) is isolated from the sample and digested with a restriction endonuclease which cleaves DNA at the site of the mutation, and cleavage of the DNA within the hnf4α P2 region at this site is determined. After digestion, the resulting DNA fragments may be subjected to electrophoresis on an agarose gel. DNA from the gel may be visualised, for instance by staining with ethidium bromide. DNA from the gel may also be blotted onto a nitrocellulose filter and hybridised with a labelled probe, such as for instance a radiolabelled probe or a probe labelled as described further below. The probe may conveniently contain a DNA fragment of the hnf4α P2 region spanning the mutation (substantially according to the method of E. M. Southern, J. Mol. Biol. 98, 503 (1975), e.g. as described by B. J. Conner et al., Proc. Natl. Acad. Sci. USA 80, 278-282 (1983)).

Digestion of the DNA may preferably be performed as recommended by the supplier of the enzyme.

In a further embodiment of this method, the restriction pattern of the DNA after digestion with the restriction endonuclease, whether visualised by staining with ethidium bromide or by hybridising with a labelled probe or otherwise, is compared to the restriction pattern obtained with a negative control comprising at least a portion of wild-type hnf4α P2 region (i.e. not containing the mutation) and/or to the restriction pattern obtained with a positive control comprising at least a portion of the hnf4α P2 region and containing the mutation.

It is a question of routine work for a person skilled in the art to determine whether DNA spanning a given mutation in the hnf4α P2 region may be cleaved by use of an restriction endonuclease and, in that case, which restriction endonuclease(s) will be suitable for the task and how to analyse the resulting restriction patterns.

In a variant of these embodiments, the DNA isolated from the sample may be amplified prior to digestion with the restriction endonuclease. Amplification may suitably be performed by polymerase chain reaction (PCR) using oligonucleotide primers based on the appropriate sequence of PTP-1B spanning the site(s) of mutation, essentially as described by Saiki et al., Science 230, 1350-1354 (1985). After amplification, the amplified DNA may be digested with the appropriate restriction endonuclease and subjected to agarose gel electrophoresis. The restriction pattern obtained may be analysed, e.g. by staining with ethidium bromide and visualising bands in the gel by means of UV light. As a control, wild-type DNA encoding PTP-1B (i.e. not containing the mutation) may be subjected to the same procedure, and the restriction patterns may be compared.

In one embodiment of a diagnostic method according to the present invention a sample nucleic acid is obtained from a subject, DNA is isolated from the sample, the DNA is amplified and hybridised to a labelled polynucleotide comprising the hnf4α P2 region, said nucleotide sequence containing the mutation of interest, or comprising a fragment of the nucleotide sequence including said mutation, which mutation corresponds to the mutation the presence of which in the hnf4α P2 region is to be detected, and hybridisation of the labelled polynucleotide to the DNA is determined.

In a further embodiment of said method, the amplified DNA is hybridised to a second labelled polynucleotide comprising a DNA sequence corresponding to at least part of the wild-type hnf4α P2 region, and hybridisation of said second labelled polynucleotide to the amplified DNA is determined.

In a further embodiment of said diagnostic method, the label substance with which the labelled polynucleotide carrying the mutation is labelled is different from the label substance with which the second labelled polynucleotide corresponding to at least part of the wild-type DNA is labelled.

Another embodiment of a diagnostic method according to the present invention is an adaptation of the method described by U. Landegren et al., Science 241, 1077-1080 (1988), which involves the ligation of adjacent oligonucleotides on a complementary target DNA molecule. Ligation will occur at the junction of the two oligonucleotides if the nucleotides are correctly base paired.

In a further embodiment of a method according to the present invention, the sample nucelic acid may be amplified using oligonucleotide primers corresponding to segments of the hnf4α P2 region. The amplified DNA may then be analysed by hybridisation with a labelled polynucleotide comprising a DNA sequence corresponding to at least part of the hnf4α P2 region and containing the mutation the presence of which in the hnf4α P2 region is to be detected. As a control, the amplified DNA may furthermore be hybridised with a further labelled polynucleotide comprising a DNA sequence corresponding to at least part of the wild-type hnf4α P2 region. This procedure is, for instance, described by DiLella et al., Lancet 1, 497-499 (1988). Another PCR-based method which may be used in the present invention is the allele-specific PCR method described by R. Saiki et al., Nature 324, 163-166 (1986), or D. Y. Wu et al., Proc. Natl. Acad. Sci. USA 86, 2757-2760 (1989), which uses primers specific for the mutation in the hnf4α P2 region.

Other methods of detecting mutations in DNA are reviewed in U. Landegren, GATA 9, 3-8 (1992) One of the currently preferred methods of detecting mutations is by single stranded conformation polymorphism (SSCP) analysis substantially as described by Orita et al., Proc. Natl. Acad. Sci. USA 86, 2766-2770 (1989), or Orita et al., Genomics 5, 874-879 (1989) and another is single base extension (also known as microsequencing) substantially as described by Syvänen, A.-C. et al., Genomics 12, 590-5 (1992).

The label substance with which a polynucleotide may be labelled may be selected from the group consisting of enzymes, coloured or fluorescent substances, or radioactive isotopes.

Examples of enzymes useful as label substances are peroxidases (such as horseradish peroxidase), phosphatases (such as acid or alkaline phosphatase), β-galactosidase, urease, glucose oxidase, carbonic anhydrase, acetylcholinesterase, glucoamylase, lysozyme, malate dehydrogenase, glucose-6-phosphate dehydrogenase, β-glucosidase, proteases, pyruvate decarboxylase, esterases, luciferase, etc.

Enzymes are not in themselves detectable but must be combined with a substrate to catalyse a reaction the end product of which is detectable. Examples of substrates, which may be employed in the method according to the invention, include hydrogen peroxide/tetramethylbenzidine or chloronaphthole or o-phenylenediamine or 3-(p-hydroxyphenyl) propionic acid or luminol, indoxyl phosphate, p-nitrophenylphosphate, nitrophenyl galactose, 4-methyl umbelliferyl-D-galactopyranoside, or luciferin.

Alternatively, the label substance may comprise coloured or fluorescent substances, including gold particles, coloured or fluorescent latex particles, dye particles, fluorescein, phycoerythrin or phycocyanin.

In one embodiment, the labelled polynucleotide is labelled with a radioactive isotope. Radioactive isotopes, which may be used for the present purpose, may be selected from I-125, I-131, In-111, H-3, P-32, C-14 or S-35. The radioactivity emitted by these isotopes may be measured in a beta- or gamma-counter or by a scintillation camera in a manner known per se.

Methods

Animal Breeding, Tissue Isolation.

A colony of hnf1α^{-/-} mice generated in the laboratory of F. Gonzalez (NIH, Bethesda, Md.) was established locally and maintained as described (Parrizas, M. et al (2001), ibid, Lee, Y. et al (1998) Mol. Cell Biol. 18, 3059-3068). Embryos from timed pregnancies were collected and used for pancreas dissection. Mouse hepatocytes, pancreatic islets and exocrine tissue were isolated as described (Parrizas, M. et al (2001), ibid).

RNA Analysis.

Total RNA and reverse transcription were performed as described (Parrizas, M. et al (2001), ibid). PCR was carried out with oligonucleotides designed to span an intron, and all reactions included controls lacking cDNA or reverse transcriptase. Test products were coamplified with an internal control (β-actin, hprt or tbp). At least two cycle numbers were tested in each experiment, and conditions were adjusted so that both products were in the exponential phase of amplification. Primer sequences and reaction conditions are available upon request. All positive results were verified in at least 5 control and mutant mice. 5′rapid amplification of cDNA ends was performed using the Marathon system (Clontech), with two sequential rounds of amplification using first external and then internal gene-specific oligonucleotides complementary to hnf4α exon 2 cDNA (5′-GGTCCCCGCAGATGGCACAC-3′ and 5′-CTGTTGGGCGCGTTGAGGTTGGT-3′) respectively.

Computer Genome Analysis

Human genome and trace mouse genome sequences were analyzed with ENSEMBL (http://www.ensembl.org/). Promoter prediction was performed with TSSW and NPPW programs using the BCM Search Launcher (Smith, R. F. et al (1996) Genome Res. 6, 454-462). Alignment was performed with CLUSTAL V using DNASTAR (Madison, Wis.).

Immunohystochemistry

Immunofluorescence analysis was performed as described (Parrizas, M. et al (2001), ibid), using three-micrometer sections from paraffin-embedded embryos dissected at E13.5, E15.5, E18.5, or 2-week-old mice.

Electromobility Shift Assays (EMSAs)

The hnf1α binding site from the human HNF4α P2 promoter was analyzed with oligonucleotides 5′-AG TGACTGGTTACTCTTTAACGTATCCAC-3′ (wild type) and 5′ AGTGACTGGTTcCTCTTgAACGTATCCAC 3′ (Mutated bases in small case). ³²P-labeled oligonucleotides were incubated 20 min at 22° C. with 5 μg nuclear extracts from total pancreas, isolated islets, hepatocytes, MIN6 cells or recombinant glutathione-S-transferase-hnf1α in 20 mM HEPES, pH 7.9, 90 mM KCl, 5 mM MgCl₂ and 0.05% NP-40, in the presence or absence of cold competitor. Supershifts were performed by adding 1 μl rabbit polyclonal antibody raised against hnf1α peptides GLIEEPTGDELPTK and EASSEPGLHEPPSPA or preimmune serum to the reaction and incubating for 15 min at 22° C. before the addition of the labeled probe. Samples were electrophoresed on a 5% acrylamide gel in 0.5×TBE buffer and autoradiographed.

Chromatin Immunoprecipitation (ChIP)

ChiP's were performed as previously described (Parrizas, M. et al (2001), ibid), except that prior to sonication, fixed cells were resuspended in denaturing buffer (2 M NaCl, 5 M urea), incubated on ice 10 min, and washed twice in PBS before resuspension in sonication buffer. Multiplex PCR conditions were adjusted to ensure non saturation kinetics and similar amplification efficiencies for all amplicons within a reaction. Primers employed were designed to amplify segments either encompassing hnf1 binding sites and/or located <50 bp from the transcription initiation site region of selected transcriptional regulatory regions. Primer sequences and amplification conditions are available upon request. PCR products were analyzed on ethidium-bromide stained 10% acrylamide gels. Each PCR reaction was performed at least twice with samples resulting from three immunoprecipitation experiments.

### EXAMPLE 1

Hnf1α Controls Expression of hnf4α Selectively in Pancreatic Cells.

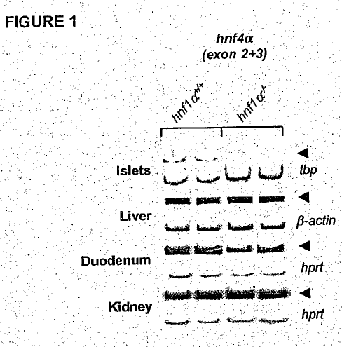

Purified pancreatic islet RNA from 3-week old hnf1α^{-/-} and hnf1α^{+/+} mice was analyzed to search for β-cell transcription factor genes downstream of hnf1α. Hnf4α, previously reported to be upstream of hnf1α in an hepatocyte transcriptional regulatory hierarchy (Kuo, C. J. et al. (1992) and Li, J. et al (2000) ibid), exhibited >15-fold decreased mRNA levels in pancreatic islets of hnf1α-deficient mice (FIG. 1). hnf4α mRNA was not affected by loss of hnf1α in liver and kidney, although it was partially inhibited in duodenum (FIG. 1). Immunohistochemical and electromobility retardation-supershift analysis of hnf4α using two different C-terminus-specific antisera in hnf1α^{-/-} and hnf1α^{+/+} adult and E15.5 embryonic pancreatic tissue revealed very weak, hnf1α-dependent hnf4α immunoreactivity under conditions that indicated strong expression in hepatocytes and kidney (not shown). This suggests that despite genetic evidence for its relevant role in pancreatic islet cells (Yamagata, K et al (1996), ibid, and Byrne, M. M. et al (1995) Diabetes 44, 699-704), the abundance of hnf4α protein in mouse pancreas is low.

### EXAMPLE 2

Pancreatic hnf4α mRNA is Transcribed from a Tissue-Specific Alternative Promoter.

One potential mechanism to diversify transcriptional control of a gene in different tissues is the utilization of alternative promoters. Although only a single hnf4α promoter has been described to date, 5′ rapid amplification of cDNA ends of hnf4α mRNA in human pancreatic islets revealed a previously unreported sequence that is 82% identical to the 5′ end of an alternatively spliced mouse hnf4α cDNA known as hnf4α7 (Nakhei, H. et al (1998), ibid). Analysis of human and mouse genome sequences revealed that this alternate 5′ leader sequence constitutes a single conserved exon in both species, which we refer to as exon 1D (FIG. 2a).

Transcripts containing the mouse exon 1D leader sequence were previously shown to be the prevalent hnf4α species in early embryonic cells, with only low levels detected in several adult tissues Nakhei, H. et al (1998). As shown FIG. 2b, nearly all adult mouse islet hnf4α transcripts contain this alternative 5′ mRNA sequence. In contrast, hnf4α transcripts in liver or kidney almost exclusively contain the exon 1A leader sequence, while both forms are expressed in duodenum. Interestingly, hnf4α transcripts containing exon 1D are present and dependent on hnf1α in both endocrine and exocrine pancreatic compartments (FIG. 2c, compare with expression patterns of islet and exocrine-specific markers). Pancreatic hnf4α transcripts containing exon 1D include 3 possible known variants in the 3′ end of the RNA (data not shown), and therefore constitute not solely hnf4α7 but also a novel combination of known splice variations referred to as hnf4α8 and hnf4α9 following an existing nomenclature (Sladek, F. M. & Senkel, S. (2001) in Nuclear receptors and genetic disease, eds. Burris T. P. & McCabe E. R. B. (Academic Press, San Diego), pp. 309-361).

Based on evidence of an alternative hnf4α mRNA initiation site in mouse and human islets, we refer to the 5′ flanking region of exon 1D as the hnf4α P2 promoter region. This region is located 45.6 Kb upstream of the previously known promoter (P1) and exon 1A (FIGS. 2a and 2d). Computer analysis of human and mouse genomic sequences using two different algorithms predicted a putative transcription start site which coincides with the reported 5′ end of mouse hnf4α7 cDNA (FIG. 2a). In vivo supportive evidence that P2 represents the active hnf4α promoter region in pancreatic cells was obtained from chromatin immunoprecipitation (ChIP) analysis of nucleosomal histone acetylation. Hyperacetylation of chromatin histone tails is thought to represent a key event in the activation of many eukaryotic genes (Struhl, K. (1998) Genes Dev. 12, 599-606). Previous work showed that hnf1α-dependent tissue-specific gene activity is tightly linked to the requirement for hnf1α to maintain localized hyperacetylation of nucleosomal histones (Parrizas, M. et al (2001) Mol. Cell Biol. 21, 3234-3243). As shown in FIG. 2e, nucleosomal histones of the P2 region are hyperacetylated in pancreatic islets (lane 1), whereas P1 histones are hypoacetylated. A similar pattern is seen in MIN6 β-cells (data not shown). In contrast, P1 rather than P2 chromatin is hyperacetylated in liver (lanes 9,10), in parallel with the observed tissue-specific transcription initiation patterns. P2 nucleosomes in islets of hnf1α-deficient mice are hypoacetylated (lane 5), in keeping with the selective reduction of hnf4α mRNA in islets of hnf1α^{-/-} mice. In aggregate, these results suggest that in vivo P2 chromatin is hyperacetylated and transcriptionally active selectively in pancreatic cells, and this is dependent on hnf1α.

### EXAMPLE 3

Hnf1α Directly Occupies and Controls the Activity of the hnf4α P2 Promoter in a Tissue-Specific Manner.

It was then investigated whether control of hnf4α by hnf1α in islet-cells is direct. A consensus hnf1 site present in the P2 5′ flanking region binds recombinant and native hnf1α in vitro (FIGS. 2a and 3a). ChIP analysis of mouse islet chromatin using anti-hnf1α antibodies revealed enrichment of hnf4α P2 chromatin as compared to P1 chromatin, indicating that hnf1α occupies the hnf4α P2 promoter in vivo in pancreatic islets (FIG. 3b, lane 1). In contrast, in liver, hnf1α predominantly occupies P1, but not P2 chromatin (FIG. 3b, lane 5). The P1 promoter contains a previously identified high-affinity hnf1 binding site (Taraviras, S. (1994) Mech. Dev. 48, 67-79), which is thought to underly dependence on hnf1β in early endodermal cells (Barbacci, E. et al (1999) Development 126, 4795-4805). Thus, hnf1α directly occupies P2 chromatin in pancreatic islets, rather than acting exclusively through intermediary factors. Furthermore, occupancy by hnf1α of either P1 or P2 regions in liver or pancreas is tightly linked to regional chromatin acetylation and transcriptional activity status, suggesting that chromatin configuration modulates access of hnf1α to its cognate DNA binding sites in vivo.

### EXAMPLE 4

Hnf1α Directly Controls hnf4γ and hnf3γ mRNA Specifically in the Pancreas.

Further analysis by RT-PCR of candidate transcription factors expressed in pancreatic cells revealed an essential role of hnf1α in maintaining the expression of hnf4γ and hnf3γ mRNA (FIG. 4A, and not shown). Hnf4γ is a nuclear receptor structurally related to hnf4α but expressed at only very low levels in the liver (Taraviras, S. et al (2000) Biochim. Biophys. Acta 1490, 21-32). Hnf4γ is enriched in islet-cells within the pancreas, but is present and exhibits hnf1α-dependence in both endocrine and exocrine compartments (FIG. 4B). Hnf4γ also displays partial hnf1α-dependence in duodenum, but not in kidney cells (FIG. 4A). The reported mouse 5′ untranslated mRNA region of hnf4γ is readily detected and dependent on hnf1α in islets (not shown). Analysis of mouse genome trace sequences identified a consensus hnf1 binding site located 120 bp 5′ of the reported murine cDNA sequence, which differs by a single nucleotide from the hnf4α P2 hnf1 binding site (not shown). ChIP experiments indicate that hnf1α directly interacts with 5′ flanking hnf4γ chromatin in mouse pancreatic islets (FIG. 4C, lane 1). In liver, where hnf4γ mRNA expression is virtually undetectable by RT-PCR, we did not detect hnf1α association with the promoter region and only trace levels of nucleosomal hyperacetylation (not shown). In contrast, the hnf4γ promoter region chromatin is hyperacetylated in wild-type islets, where hnf4γ is expressed, but hypoacetylated in hnf1α^{-/-} islets, which do not express hnf4γ mRNA (FIG. 4D).

The Analysis of the Forkhead Homolog hnf3γ in hnf1α^{-/-} Pancreas

Hnf3γ mRNA exhibits hnf1α-dependence in pancreatic cells, but not in liver or duodenum where it is also expressed (FIG. 4a). Hnf3γ mRNA is clearly enriched in the exocrine pancreatic compartment, but is also represented in islet and MIN6 β-cells (FIG. 4b, see pancreatic fraction-specific markers in FIG. 2b). As shown in FIG. 4c, hnf1α occupies the hnf3γ enhancer chromatin segment in vivo both in islets and liver (lanes 5 and 13).

### EXAMPLE 5

The Pancreatic hnf1α-Dependent Genetic Program Operates in Differentiated Cells.

A fundamental question concerning the obligate role of hnf1α in pancreatic β-cell transcription lies in determining the time in development at which it is established. Hnf1α is broadly expressed in most cells of the developing pancreas as early as E13.5 (unpublished results, Miguel A. Maestro and Jorge Ferrer, and FIG. 5a), a stage in which the pancreas is populated primarily by undifferentiated epithelial cells. Hnf4α transcripts containing predominantly exon 1D are expressed in E13.5 pancreas, but similar steady state levels were found in hnf1α^{-/-} and ^{+/+}13.5 embryos. Decreased hnf4α mRNA levels are not elicited in hnf1^{-/-} embryos until E15.5-E18 (FIG. 5a). Although a moderate reduction of hnf4γ mRNA is already observed in E13.5 hnf1α^{-/-} embryos, this becomes much more pronounced a later stages of development (FIG. 5a). Hnf3γ exhibits a nearly identical pattern of hnf1α-dependence as hnf4α (FIG. 5a). The finding that hnf4α, -4γ, and -3γ mRNA is reduced in hnf1α^{-/-} embryos suggests that a) the defect is not secondary to the diabetic environment, as heterozygous mothers are normoglycemic, and b) hnf1α-dependence is elicited in parallel with the major surge of postmitotic differentiated endocrine and exocrine pancreatic cells occurring between E14 and E17 (Pang, K. et al (1994) Proc. Natl. Acad. Sci. U.S.A 91, 9559-9563 and Slack, J. M. (1995) Development 121, 1569-1580).

Whether the loss of intermediary transcription factors during pancreatic development correlates with the establishment of defective expression of two genes, glut2 and pklr, which in adult tissues are dependent on hnf1α selectively in pancreatic islet-cells (Parrizas, M. et al (2001), ibid), was then assessed. Glut2 expression is largely restricted to β-cells within the adult pancreas, although it is known that early multipotent pancreatic epithelial cells also express glut2 (Pang, K. et al (1994)). Pancreatic mRNA levels of glut2 and pklr are similar in control and hnf1^{-/-} E13.5 embryos, but then decrease between E15.5 and E18.5 (FIG. 5b). In keeping with these findings, glut2 immunostaining is unaltered in hnf1α^{-/-} early pancreatic epithelial cells (FIG. 5c, left panels). At embryonic day E15.5, shortly after the major surge of insulin-producing cells arises, most glut2-positive cells in control mice still represent a precursor pool not expressing endocrine differentiation markers, but all insulin-positive cells exhibit glut2 staining (FIG. 5c, middle upper panel, and data not shown). In E15.5 hnf1α^{-/-} embryos only some insulin-positive cells express glut2 at varying degrees, whereas many others do not exhibit glut2 immunoreactivity (FIG. 5c, middle lower panel). From this point on, glut2 expression is lost in a progressively larger fraction of β-cells, and is uniformly undetectable in hnf1α^{-/-} mice by embryonic day E18.5 (FIG. 5c, right panels, and data not shown for E16.5). These findings indicate that while hnf1α is coexpressed with glut2 in multiple early pancreatic cell types, it is indispensable for glut2 expression only after insulin-producing cells initiate terminal differentiation, in parallel with its requirement to maintain pancreatic expression of hnf4α, hnf4γ and hnf3γ.

1. A method for screening for modulators of expression of hnf4α from the hnf4α P2 promoter region, said method comprising the steps of
a) contacting a putative modulator with a transcription reporter system comprising a reporter gene under control of an hnf4α P2 promoter region corresponding to nucleotides 1 to 1447 of SEQ ID No. 1 or a fragment of nucleotides 1 to 1447 of SEQ ID No. 1, and
b) assaying for a change in expression of the reporter gene.

2. The screening method according to claim wherein the fragment of nucleotides 1 to 1447 of SEQ ID No. 1 comprises an hnf1α binding site.

3. The method according to claim 2, wherein the hnf1α binding site comprises nucleotides 79 to 93 of SEQ ID No. 3 or a fragment thereof to which hnf1α is capable of binding.

4. The method according to claim 1 wherein the change in expression of the reporter gene is an increase in expression.

5. A modulator of expression of hnf4α identified by a method according to claim 1.

6. A method for treating diabetes in an animal, said method comprising the step of modulating the expression of hnf4α in pancreatic cells by administering to said animal an effective dose of a modulator according to claim 5.

7. The method according to claim 6 wherein the modulation is effected via the hnf4α P2 promoter corresponding to nucleotides 1 to 1447 of SEQ ID No. 1.

8. The method according to claim 6, wherein administration of the modulator results in an increase in expression of hnf4α.

9. The method according to claim 6, wherein the modulation is effected via a hnf1α binding site corresponding to nucleotides 79 to 93 of SEQ ID No. 3 or a fragment thereof to which hnf1α is capable of binding.

10. The method according to 6, wherein the animal is a mammal.

11. The method according to claim 10, where the mammal is a human being.

12. The method according to claim 6, wherein the animal suffers from maturity onset diabetes of the young (MODY).

13. The method according to claim 12, wherein the animal suffers from MODY1.

14. The method according to claim 6, wherein the animal suffers from type 2 diabetes.

15. Use of a modulator according to claim 5 for treating diabetes in an animal.

16. Use of a modulator according to claim 5 for treating MODY in an animal.

17. Use of a modulator according to claim 5 for treating type 2 diabetes in an animal.

18. Use according to any of claims 15 to 17 wherein the animal is a mammal.

19. Use according to claim 18 wherein the animal is a human being.

20. A method for screening for diabetes in an animal, said method comprising:
analyzing a nucleic acid molecule obtained from said animal to detect a mutation in the hnf4α P2 promoter region as shown in SEQ ID No. 1, wherein the mutation results in a lesser degree of expression of hnf4α and, is indicative of a propensity for diabetes.

21. The method according to claim 20, wherein the hnf4α P2 promoter region corresponds to nucleotides 1 to 1447 of SEQ ID No. 1.

22. The method according to claim 20 wherein the hnf4α P2 promoter region comprises the region corresponding to nucleotides 79 to 93 of SEQ ID No. 1.

23. The method according to claim 22, wherein the mutation is a mutation of a G to an A in the position corresponding to position 80 in SEQ ID No. 3.
